(19) United States
(12) Patent Application Publication
(10) Pub. No.: US 2022/0370486 A1
(43) Pub. Date: Nov. 24, 2022

(54) COMBINATION TREATMENT OF SYSTEMIC FUNGAL INFECTIONS

(71) Applicant: The Board of Trustees of the University of Illinois, Urbana, IL (US)

(72) Inventors: Martin D. Burke, Champaign, IL (US); Chad M. Rienstra, Sun Prairie, WI (US); Jiabao Zhang, Urbana, IL (US)

(21) Appl. No.: 17/633,327

(22) PCT Filed: Aug. 7, 2020
(86) PCT No.: PCT/US20/45387

§ 371 Date: Feb. 7, 2022

Related U.S. Application Data
(60) Provisional application No. 62/929,224, filed on Nov. 1, 2019.
(60) Provisional application No. 62/884,985, filed on Aug. 9, 2019.

(51) Int. Cl. A61K 31/7042; A61K 31/4164; A61K 9/00; A61K 31/425; A61P 31/10

(52) U.S. Cl. CPC A61K 31/7042; A61K 31/4164; A61K 9/0019; A61K 31/425; A61P 31/10

(57) ABSTRACT

Disclosed are compositions comprising a polyene macrolide antibiotic and an azole antifungal, and packaged pharmaceutical products comprising the disclosed compositions. Also disclosed are methods of treating systemic fungal infections comprising co-administering to a mammal a polyene macrolide antibiotic and an azole antifungal (conjoint administration). The co-administration may be simultaneous (e.g., in a single formulation or in separate formulations), sequential, or staggered.

### CROSS-REFERENCE TO RELATED APPLICATIONS

This application claims the benefit of priority to U.S. Provisional Patent Application No. 62/884,985, filed Aug. 9, 2019, and U.S. Provisional Patent Application No. 62/929,224, filed Nov. 1, 2019. The content of these applications is incorporated herein by reference in its entirety.

### GOVERNMENT SUPPORT

This invention was made with government support under grant AI135812 awarded by the National Institutes of Health. The government has certain rights in the invention.

### BACKGROUND OF THE INVENTION

Morbidity and mortality from invasive fungal infections are significant, and largely caused by two genera of fungal pathogens: Candida and Aspergillus. Candida species are the 4th most common pathogen isolated in all bloodstream infections. Treatment for invasive candidiasis has a limited (50-70%) success rate, and this is typically only in the healthiest patients. Attributable mortality for invasive candidiasis is substantial (20-30%). The incidence of invasive aspergillosis due to A. fumigatus has increased three-fold in the last decade and its mortality has risen by over 300%. Moreover, current therapy for invasive aspergillosis has a lower 40-50% treatment success rate. Invasive aspergillosis is consistently a leading killer in immunocompromised patients, and moreover, whereas invasive mold infections (fusariosis, scedosporosis, and mucromycosis) have even higher mortality rates and no effective therapeutic options. The current guideline-recommended first line therapeutic for invasive aspergillosis, as well as most other invasive mold infections, is the triazole antifungal voriconazole. However, pan-triazole resistance in Aspergillus is as high as 30% in some locations and amongst certain high-risk patient groups. Recognizing this lack of effective treatments, the Infectious Diseases Society of America highlighted A. fumigatus as one of only six pathogens where a “substantive breakthrough is urgently needed.”

Amphotericin B (AmB) is an exceptionally promising starting point, because this drug has potent and dose-dependent fungicidal activity against a broad range of fungal pathogens and has evaded resistance for over half a century The fungicidal, as opposed to fungistatic, activity of AmB is essential in immunocompromised patients which lack a robust immune system to help clear an infection. Broad antifungal activity is especially important in critically ill patients when the identity of the pathogen is unknown and immediate empirical therapy is required. An international expert panel recently mandated that novel therapeutic approaches centered around AmB, with no resistance issues, are required. The problem is that AmB is exceptionally toxic, which limits its use to low-dose protocols that often fail to eradicate disease.

A new, paradigm-shifting mechanistic understanding of AmB that evaded the field for half a century was achieved. Previous studies report AmB binding to sterols, which was such thought to primarily drive formation of membrane-permeabilizing pores to kill both fungal and human cells. After 10 years of intensive synthesis-enabled atomistic interrogations of this natural product and frontier SSNMR experiments, it is alternatively discovered that AmB primarily kills both fungal and human cells by forming a cytocidal extramembranous sterol sponge. This large aggregate sits on the surface of lipid bilayers and rapidly extracts membrane sterols, which leads to cell death. Membrane permeabilization is not required. Based on this new mechanism and increasingly refined structural information, it is proposed that a small molecule-based ligand-selective allosteric effect could enable selective binding of ergosterol over cholesterol. Guided by this model, the elimination of cholesterol binding and thus mammalian toxicity in the form of a new derivative, C2′epiAmB, was achieved.

A limitation with C2′epiAmB, however, is lack of potency against a number of clinically relevant yeast and molds.

### SUMMARY OF THE INVENTION

In certain aspects, provided herein are pharmaceutical composition, comprising a polyene macrolide antibiotic; and an azole antifungal compound; wherein the azole antifungal compound inhibits lanosterol 14-alpha demethylase.

In certain embodiments, the azole antifungal compound is an imidazole, a triazole, or a thiazole. In further embodiments, the azole antifungal compound is an imidazole. In yet further embodiments, the imidazole is selected from the group consisting of bifonazole, butoconazole, clotrimazole, econazole, fenticonazole, isoconazole, ketoconazole, luliconazole, miconazole, omoconazole, oxiconazole, sertaconazole, sulconazole, and tioconazole.

In certain embodiments, the azole antifungal compound is a triazole. In further embodiments, the triazole is selected from the group consisting of albaconazole, efinaconazole, epoxiconazole, fluconazole, isavuconazole, itraconazole, posaconazole, propiconazole, ravuconazole, terconazole, and voriconazole. In yet further embodiments, the azole antifungal compound is a thiazole. In still further embodiments, the thiazole is abafungin. In still further embodiments, the polyene macrolide antibiotic is selected from the group consisting of amphotericin B, C2′epi amphotericin B, candicidin, filipin, hamycin, natamycin, nystatin, and rimocidin.

In certain embodiments, the polyene macrolide antibiotic is amphotericin B. In certain embodiments, the polyene macrolide antibiotic is C2′epi amphotericin B.

In certain embodiments, the composition is an intravenous dosage form. In further embodiments, the composition is an oral dosage form. In yet further embodiments, the oral dosage form is a tablet. In still further embodiments, the oral dosage form is a capsule.

In further aspects, provided herein are methods of treating a systemic fungal infection, comprising co-administering to a mammal in need thereof an effective amount of a polyene macrolide antibiotic; and an effective amount of an azole antifungal compound; wherein the azole antifungal compound inhibits lanosterol 14-alpha demethylase.

In certain embodiments, the azole antifungal compound is an imidazole, a triazole, or a thiazole. In further embodiments, the azole antifungal compound is an imidazole. In yet further embodiments, the imidazole is selected from the group consisting of bifonazole, butoconazole, clotrimazole, econazole, fenticonazole, isoconazole, ketoconazole, luliconazole, miconazole, omoconazole, oxiconazole, sertaconazole, sulconazole, and tioconazole.

In certain embodiments, the azole antifungal compound is a triazole. In further embodiments, the triazole is selected from the group consisting of albaconazole, efinaconazole, epoxiconazole, fluconazole, isavuconazole, itraconazole, posaconazole, propiconazole, ravuconazole, terconazole, and voriconazole. In yet further embodiments, the azole antifungal compound is a thiazole. In certain embodiments, the thiazole is abafungin.

In certain embodiments, the polyene macrolide antibiotic is selected from the group consisting of amphotericin B, C2′epi amphotericin B, candicidin, filipin, hamycin, natamycin, nystatin, and rimocidin.

In certain embodiments, the polyene macrolide antibiotic is amphotericin B. In certain embodiments, the polyene macrolide antibiotic is C2′epi amphotericin B.

In certain embodiments, the polyene macrolide antibiotic and the azole antifungal compound are administered in separate dosage forms. In further embodiments, the polyene macrolide antibiotic and the azole antifungal compound are administered simultaneously, sequentially, or intermittently. In yet further embodiments, the polyene macrolide antibiotic and the azole antifungal compound are administered simultaneously. In still further embodiments, the polyene macrolide antibiotic and the azole antifungal compound are administered sequentially. In certain embodiments, the polyene macrolide antibiotic and the azole antifungal compound are administered intermittently.

In certain embodiments, the polyene macrolide antibiotic and the azole antifungal compound are administered in a combined dosage form. In further embodiments, the combined dosage form is administered intravenously. In yet further embodiments, the combined dosage form is administered orally.

In yet further aspects, provided herein are packaged pharmaceutical products, comprising a polyene macrolide antibiotic; and an azole antifungal compound; wherein the azole antifungal compound inhibits lanosterol 14-alpha demethylase.

In certain embodiments, the azole antifungal compound is an imidazole, a triazole, or a thiazole. In further embodiments, the azole antifungal compound is an imidazole. In yet further embodiments, the imidazole is selected from the group consisting of bifonazole, butoconazole, clotrimazole, econazole, fenticonazole, isoconazole, ketoconazole, luliconazole, miconazole, omoconazole, oxiconazole, sertaconazole, sulconazole, and tioconazole.

In certain embodiments, the azole antifungal compound is a triazole. In further embodiments, the triazole is selected from the group consisting of albaconazole, efinaconazole, epoxiconazole, fluconazole, isavuconazole, itraconazole, posaconazole, propiconazole, ravuconazole, terconazole, and voriconazole. In yet further embodiments, the azole antifungal compound is a thiazole. In still further embodiments, the thiazole is abafungin.

In certain embodiments, the polyene macrolide antibiotic is selected from the group consisting of amphotericin B, C2′epi amphotericin B, candicidin, filipin, hamycin, natamycin, nystatin, and rimocidin. In certain embodiments, the polyene macrolide antibiotic is amphotericin B. In certain embodiments, the polyene macrolide antibiotic is C2′epi amphotericin B.

In certain embodiments, the polyene macrolide antibiotic and the azole antifungal compound are in separate dosage forms. In further embodiments, the polyene macrolide antibiotic and the azole antifungal compound are in a combined dosage form. In yet further embodiments, the combined dosage form is an intravenous dosage form. In still further embodiments, the combined dosage form is an oral dosage form.

### BRIEF DESCRIPTION OF THE FIGURES

FIG. 1A-1C depict a ligand-selective allosteric effects model, along with the rational design of C2′epiAmB.

FIG. 2A-2D depict the sterol binding selectivity for AmB and C2′epiAmB.

FIG. 3A-3B depict the binding and toxic concentrations of AmB, AmdeB, and C2′epiAmB in cholesterol and ergosterol-containing cells.

FIG. 4A-4B depict the toxicity of AmB and C2′epiAmB in mice and rats.

FIG. 5 depicts the decreased toxicity of C2′epiAmB compared to Ambisome in mice.

FIG. 6A-6D depict the efficacy of C2′epiAmB for different pathogens.

FIG. 7A-7B depict the efficacy of C2′epiAmB in invasive candidiasis model in mice: Candida albicans SN250: MIC AmB 0.15 C2′epiAmB 0.5 μM..

FIG. 8 depicts the possible mechanisms for the decreased potentcy of C2′epiAmB.

FIG. 9A-9B depict plots and tables demonstrating that the decreased potentcy of C2′AmB in vivo is likely not due to decreased cell wall penetrance, decreased membrane permeabilization, or drug deactivation.

FIG. 10 depicts killing kinetics for AmB and C2′epiAmB against CA SN250.

FIG. 11A-11B depict the relative rates of ergosterol extraction and killing for AmB and C2′epiAmB.

FIG. 12 depicts the rates of ergosterol biosynthesis vs. extraction.

FIG. 13 depicts killing kinetics against C. Albicans SN250 for AmB, ketoconazole, C2′epiAmB, and a combination of C2′epiAmB and ketoconazole.

### DETAILED DESCRIPTION OF THE INVENTION

Amphotericin B (AmB) is a polyene macrolide with a mycosamine appendage, the complete compound has the structure below.

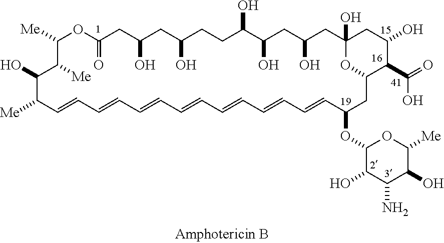

AmB is generally obtained from a strain of Streptomyces nodosus. It is currently approved for clinical use in the United States for the treatment of progressive, potentially life-threatening fungal infections, including such infections as systemic or deep tissue candidiasis, aspergillosis, cryptococcosis, blastomycosis, coccidioidomycosis, histoplasmosis, and mucormycosis, among others. It is generally formulated for intravenous injection.

Amphotericin B is commercially available, for example, as Fungizone® (Squibb), Amphocin® (Pfizer), Abelcet® (Enzon), and Ambisome® (Astellas). Due to its undesirable toxic side effects, dosing is generally limited to a maximum of about 1.0 mg/kg/day and total cumulative doses not to exceed about 3 g in humans.

AmB kills both fungal and human cells by forming a cytocidal extramembranous sterol sponge. Anderson, T. M. et al., Nat Chem Biol 2014, 10 (5), 400-6. This large aggregate sits on the surface of lipid bilayers and rapidly extracts membrane sterols, which leads to cell death. Membrane permeabilization is not required. Based on this mechanism, a small molecule-based ligand-selective allosteric effect would enable selective binding of ergosterol over cholesterol and would eliminate the mammalian toxicity of AmB (in the form of C2′epiAmB). See Wilcock, B. C. et al., J Am Chem Soc 2013, 135 (23), 8488-91. The present invention discloses the K_{DS} for the binding of both ergosterol and cholesterol to the AmB sterol sponge, which provides a quantitative and mechanistically-grounded biophysical parameter to guide rational optimization of the therapeutic index of this clinically significant natural product.

Of particular interest is the C2′-epimer of AmB (C2′epi AmB). The structure of C2′-epi AmB is shown below.

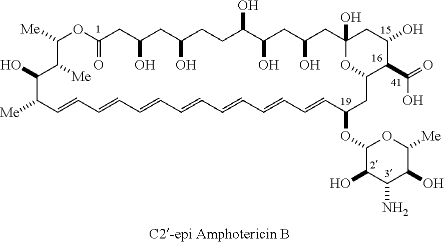

Also of interest are the polyene macrolide antibiotics candicidin, filipin, hamycin, natamycin, nystatin, and rimocidin.

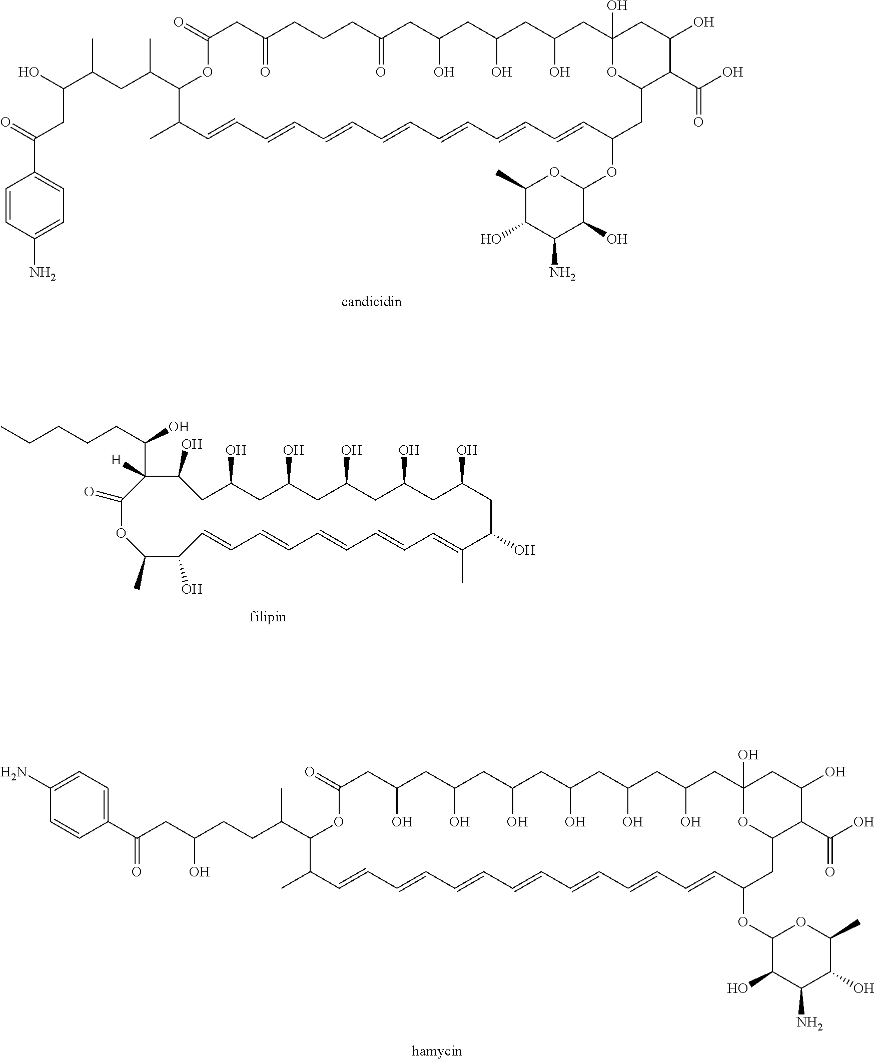 [SMILES: CC1/C=C/C=C/C=C/C=C/C=C/C=C/C=C/C(OC2O[C@H](C)[C@@H](O)[C@H](N)[C@@H]2O)CC2OC(O)(CC(O)CC(O)CC(O)CC(=O)CCCC(=O)CC(=O)OC1C(C)CC(C)C(O)CC(=O)c1ccc(N)cc1)CC(O)C2C(=O)O ; CCCCC[C@@H](O)[C@H]1C(=O)O[C@H](C)[C@@H](O)/C=C/C=C/C=C/C=C/C=C(\C)[C@@H](O)C[C@H](O)C[C@H](O)C[C@H](O)C[C@H](O)C[C@H](O)C[C@@H]1O ; CC1/C=C/C=C/C=C/C=C/C=C/C=C/C=C/C(OC2O[C@@H](C)[C@H](O)[C@@H](N)[C@H]2O)CC2OC(O)(CC(O)CC(O)CC(O)CC(O)CC(O)CC(O)CC(=O)OC1C(C)CCC(O)CC(=O)c1ccc(N)cc1)CC(O)C2C(=O)O]

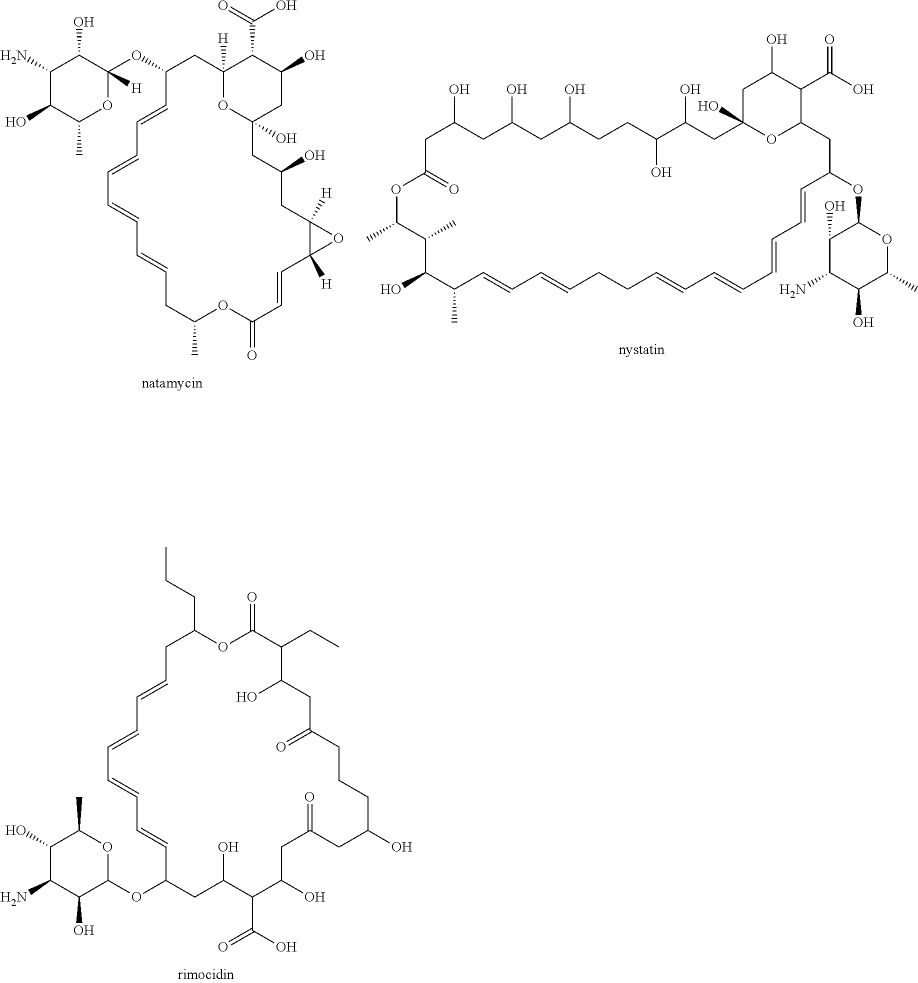

### Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

The term “pharmaceutically acceptable cation” refers to an acceptable cationic counterion of an acidic functional group. Such cations are exemplified by sodium, potassium, calcium, magnesium, ammonium, tetraalkylammonium cations, and the like (see, e. g., Berge, et al., J. Pharm. Sci. 66 (1):1-79 (January 77).

“Pharmaceutically acceptable vehicle” refers to a diluent, adjuvant, excipient or carrier with which a compound of the invention is administered.

“Pharmaceutically acceptable metabolically cleavable group” refers to a group which is cleaved in vivo to yield the parent molecule of the structural formula indicated herein. Examples of metabolically cleavable groups include —COR, —COOR, —CONRR and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkylesters or (alkoxycarbonyl)oxy)alkylesters. Particularly the C¹-8 alkyl, C2-8 alkenyl, C2-8 alkynyl, aryl, C7-12 substituted aryl, and C7-12 arylalkyl esters of the compounds of the invention.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” to which administration is contemplated includes, but is not limited to, humans (i.e., a male or female of any age group, e.g., a pediatric subject (e.g, infant, child, adolescent) or adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein. An “effective amount” means the amount of a compound that, when administered to a subject for treating or preventing a disease, is sufficient to effect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“Preventing” or “prevention” or “prophylactic treatment” refers to a reduction in risk of acquiring or developing a disease or disorder (i.e., causing at least one of the clinical symptoms of the disease not to develop in a subject not yet exposed to a disease-causing agent, or predisposed to the disease in advance of disease onset.

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be “²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radio-active isotopes tritium, i.e., ³H, and carbon-14, i.e., ¹⁴C, are particularly useful for this purpose in view of their ease of incorporation and ready means of detection. Further, com pounds may be prepared that are substituted with positron emitting isotopes, such as ¹¹C, ¹⁸F, ¹⁵O and ¹³N, a N, and would be useful in Positron Emission Topography (PET) studies for examining substrate receptor occupancy. All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R - and S -sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 95% by weight R-compound and at most about 5% by weight S-compound, at least about 99% by weight R-compound and at most about 1% by weight S-compound, or at least about 99.9% by weight R-compound and at most about 0.1% by weight S-compound. In certain embodiments, the weights are based upon total weight of compound.

As used herein and unless otherwise indicated, the term “enantiomerically pure S-compound” or “S-compound” refers to at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)- stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

One having ordinary skill in the art of organic synthesis will recognize that the maximum number of heteroatoms in a stable, chemically feasible heterocyclic ring, whether it is aromatic or non-aromatic, is determined by the size of the ring, the degree of unsaturation and the valence of the heteroatoms. In general, a heterocyclic ring may have one to four heteroatoms so long as the heteroaromatic ring is chemically feasible and stable.

### Pharmaceutical Compositions

The invention also provides pharmaceutical compositions and methods for making same.

In certain aspects, provided herein are pharmaceutical composition, comprising a polyene macrolide antibiotic; and an azole antifungal compound; wherein the azole antifungal compound inhibits lanosterol 14-alpha demethylase.

In certain embodiments, the azole antifungal compound is an imidazole, a triazole, or a thiazole. In further embodiments, the azole antifungal compound is an imidazole. In yet further embodiments, the imidazole is selected from the group consisting of bifonazole, butoconazole, clotrimazole, econazole, fenticonazole, isoconazole, ketoconazole, luliconazole, miconazole, omoconazole, oxiconazole, sertaconazole, sulconazole, and tioconazole.

In certain embodiments, the azole antifungal compound is a triazole. In further embodiments, the triazole is selected from the group consisting of albaconazole, efinaconazole, epoxiconazole, fluconazole, isavuconazole, itraconazole, posaconazole, propiconazole, ravuconazole, terconazole, and voriconazole. In yet further embodiments, the azole antifungal compound is a thiazole. In still further embodiments, the thiazole is abafungin. In still further embodiments, the polyene macrolide antibiotic is selected from the group consisting of amphotericin B, C2′epi amphotericin B, candicidin, filipin, hamycin, natamycin, nystatin, and rimocidin.

In certain embodiments, the polyene macrolide antibiotic is amphotericin B. In certain embodiments, the polyene macrolide antibiotic is C2′epi amphotericin B.

In certain embodiments, the composition is an intravenous dosage form. In further embodiments, the composition is an oral dosage form. In yet further embodiments, the oral dosage form is a tablet. In still further embodiments, the oral dosage form is a capsule.

An aspect of the invention is a pharmaceutical composition comprising a compound of the invention; and a pharmaceutically acceptable carrier. In certain embodiments, the invention is a pharmaceutical composition, comprising a compound of the invention, or a pharmaceutically acceptable salt thereof, and a pharmaceutically acceptable carrier. The term “pharmaceutically acceptable carrier” means one or more compatible solid or liquid filler, diluent, or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficacy.

In certain embodiments, the pharmaceutical composition is an intravenous dosage form.

In certain embodiments, the pharmaceutical composition is an oral dosage form.

In certain embodiments, the pharmaceutical composition is a lyophilized preparation of a liposome-intercalated or liposome-encapsulated active compound.

In certain embodiments, the pharmaceutical composition is a lipid complex of the compound in aqueous suspension.

The foregoing embodiments of pharmaceutical compositions of the invention are meant to be exemplary and are not limiting.

Also provided is a method for making such pharmaceutical compositions. The method comprises placing a compound of the invention, or a pharmaceutically acceptable salt thereof, in a pharmaceutically acceptable carrier.

### Methods of the Invention

In further aspects, provided herein are methods of treating a systemic fungal infection, comprising co-administering to a mammal in need thereof an effective amount of a polyene macrolide antibiotic; and an effective amount of an azole antifungal compound; wherein the azole antifungal compound inhibits lanosterol 14-alpha demethylase.

In certain embodiments, the azole antifungal compound is an imidazole, a triazole, or a thiazole. In further embodiments, the azole antifungal compound is an imidazole. In yet further embodiments, the imidazole is selected from the group consisting of bifonazole, butoconazole, clotrimazole, econazole, fenticonazole, isoconazole, ketoconazole, luliconazole, miconazole, omoconazole, oxiconazole, sertaconazole, sulconazole, and tioconazole.

In certain embodiments, the azole antifungal compound is a triazole. In further embodiments, the triazole is selected from the group consisting of albaconazole, efinaconazole, epoxiconazole, fluconazole, isavuconazole, itraconazole, posaconazole, propiconazole, ravuconazole, terconazole, and voriconazole. In yet further embodiments, the azole antifungal compound is a thiazole. In certain embodiments, the thiazole is abafungin.

In certain embodiments, the polyene macrolide antibiotic is selected from the group consisting of amphotericin B, C2′epi amphotericin B, candicidin, filipin, hamycin, natamycin, nystatin, and rimocidin.

In certain embodiments, the polyene macrolide antibiotic is amphotericin B. In certain embodiments, the polyene macrolide antibiotic is C2′epi amphotericin B.

In certain embodiments, the polyene macrolide antibiotic and the azole antifungal compound are administered in separate dosage forms. In further embodiments, the polyene macrolide antibiotic and the azole antifungal compound are administered simultaneously, sequentially, or intermittently. In yet further embodiments, the polyene macrolide antibiotic and the azole antifungal compound are administered simultaneously. In still further embodiments, the polyene macrolide antibiotic and the azole antifungal compound are administered sequentially. In certain embodiments, the polyene macrolide antibiotic and the azole antifungal compound are administered intermittently.

In certain embodiments, the polyene macrolide antibiotic and the azole antifungal compound are administered in a combined dosage form. In further embodiments, the combined dosage form is administered intravenously. In yet further embodiments, the combined dosage form is administered orally.

Compositions of the invention are useful for inhibiting growth of fungi and yeast, including, in particular, fungi and yeast of clinical significance as pathogens. Compositions of the invention are useful in methods of treating fungal and yeast infections, including, in particular, systemic fungal and yeast infections. Compositions of the invention are also useful in the manufacture of medicaments for treating fungal and yeast infections, including, in particular, systemic fungal and yeast infections. The invention further provides the use of compositions of the invention for the treatment of fungal and yeast infections, including, in particular, systemic fungal and yeast infections.

An aspect of the invention is a method of treating a fungal infection, comprising administering to a subject in need thereof a therapeutically effective amount of a compound of the invention, thereby treating the fungal infection.

As used herein, “inhibit” or “inhibiting” means reduce by an objectively measureable amount or degree compared to control. In one embodiment, inhibit or inhibiting means reduce by at least a statistically significant amount compared to control. In one embodiment, inhibit or inhibiting means reduce by at least 5 percent compared to control. In various individual embodiments, inhibit or inhibiting means reduce by at least 10, 15, 20, 25, 30, 33, 40, 50, 60, 67, 70, 75, 80, 90, or 95 percent (%) compared to control.

As used herein, the terms “treat” and “treating” refer to performing an intervention that results in (a) preventing a condition or disease from occurring in a subject that may be at risk of developing or predisposed to having the condition or disease but has not yet been diagnosed as having it; (b) inhibiting a condition or disease, e.g., slowing or arresting its development; or (c) relieving or ameliorating a condition or disease, e.g., causing regression of the condition or disease. In one embodiment the terms “treating” and “treat” refer to performing an intervention that results in (a) inhibiting a condition or disease, e.g., slowing or arresting its development; or (b) relieving or ameliorating a condition or disease, e.g., causing regression of the condition or disease. For example, in one embodiment the terms “treating” and “treat” refer to performing an intervention that results in (a) inhibiting a fungal infection, e.g., slowing or arresting its development; or (b) relieving or ameliorating a fungal infection, e.g., causing regression of the fungal infection.

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

A “fungal infection” as used herein refers to an infection in or of a subject with a fungus as defined herein. In one embodiment the term “fungal infection” includes a yeast infection. A “yeast infection” as used herein refers to an infection in or of a subject with a yeast as defined herein.

As used herein, a “subject” refers to a living mammal. In various embodiments a subject is a non-human mammal, including, without limitation, a mouse, rat, hamster, guinea pig, rabbit, sheep, goat, cat, dog, pig, horse, cow, or non-human primate. In one embodiment a subject is a human.

As used herein, a “subject having a fungal infection” refers to a subject that exhibits at least one objective manifestation of a fungal infection. In one embodiment a subject having a fungal infection is a subject that has been diagnosed as having a fungal infection and is in need of treatment thereof. Methods of diagnosing a fungal infection are well known and need not be described here in any detail.

As used herein, a “subject having a yeast infection” refers to a subject that exhibits at least one objective manifestation of a yeast infection. In one embodiment a subject having a yeast infection is a subject that has been diagnosed as having a yeast infection and is in need of treatment thereof. Methods of diagnosing a yeast infection are well known and need not be described here in any detail.

In certain embodiments, the compound is administered intravenously.

In certain embodiments, the compound is administered orally.

In certain embodiments, the compound is administered systemically.

In certain embodiments, the compound is administered parenterally.

In certain embodiments, the compound is administered intraperitoneally.

In certain embodiments, the compound is administered enterally.

In certain embodiments, the compound is administered intraocularly.

In certain embodiments, the compound is administered topically.

Additional routes of administration of compounds of the invention are contemplated by the invention, including, without limitation, intravesicularly (urinary bladder), pulmonary, and intrathecally.

As used herein, the phrase “effective amount” refers to any amount that is sufficient to achieve a desired biological effect.

As used herein, the phrase “therapeutically effective amount” refers to an amount that is sufficient to achieve a desired therapeutic effect, e.g., to treat a fungal or yeast infection.

For any compound described herein, a therapeutically effective amount can, in general, be initially determined from in vitro studies, animal models, or both in vitro studies and animal models. In vitro methods are well known and can include determination of minimum inhibitory concentration (MIC), minimum fungicidal concentration (MFC), concentration at which growth is inhibited by 50 percent (IC₅₀), concentration at which growth is inhibited by 90 percent (IC₉₀), and the like. A therapeutically effective amount can also be determined from human data for compounds of the invention which have been tested in humans and for compounds which are known to exhibit similar pharmacological activities, such as other related active agents (e.g., AmB). Higher doses may be required for parenteral administration. The applied dose can be adjusted based on the relative bioavailability and potency of the administered compound. Adjusting the dose to achieve maximal efficacy based on the methods described herein and other methods as are well-known in the art is well within the capabilities of the ordinarily skilled artisan.

For any compound described herein, a therapeutically effective amount for use in human subjects can be initially determined from in vitro studies, animal models, or both in vitro studies and animal models. A therapeutically effective amount for use in human subjects can also be determined from human data for compounds of the invention which have been tested in humans and for compounds which are known to exhibit similar pharmacological activities, such as other related active agents (e.g., AmB). Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

### Packaged Pharmaceutical Products

In yet further aspects, provided herein are packaged pharmaceutical products, comprising a polyene macrolide antibiotic; and an azole antifungal compound; wherein the azole antifungal compound inhibits lanosterol 14-alpha demethylase.

In certain embodiments, the azole antifungal compound is an imidazole, a triazole, or a thiazole. In further embodiments, the azole antifungal compound is an imidazole. In yet further embodiments, the imidazole is selected from the group consisting of bifonazole, butoconazole, clotrimazole, econazole, fenticonazole, isoconazole, ketoconazole, luliconazole, miconazole, omoconazole, oxiconazole, sertaconazole, sulconazole, and tioconazole.

In certain embodiments, the azole antifungal compound is a triazole. In further embodiments, the triazole is selected from the group consisting of albaconazole, efinaconazole, epoxiconazole, fluconazole, isavuconazole, itraconazole, posaconazole, propiconazole, ravuconazole, terconazole, and voriconazole. In yet further embodiments, the azole antifungal compound is a thiazole. In still further embodiments, the thiazole is abafungin.

In certain embodiments, the polyene macrolide antibiotic is selected from the group consisting of amphotericin B, C2′epi amphotericin B, candicidin, filipin, hamycin, natamycin, nystatin, and rimocidin. In certain embodiments, the polyene macrolide antibiotic is amphotericin B. In certain embodiments, the polyene macrolide antibiotic is C2′epi amphotericin B.

In certain embodiments, the polyene macrolide antibiotic and the azole antifungal compound are in separate dosage forms. In further embodiments, the polyene macrolide antibiotic and the azole antifungal compound are in a combined dosage form. In yet further embodiments, the combined dosage form is an intravenous dosage form. In still further embodiments, the combined dosage form is an oral dosage form.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of active compounds will be, for human subjects, from about 0.01 milligrams/kg per day to 1000 milligrams/kg per day. It is expected that oral doses in the range of 0.5 to 50 milligrams/kg, in one or several administrations per day, will yield the desired results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

In one embodiment, intravenous administration of a compound of the invention may typically be from 0.1 mg/kg/day to 20 mg/kg/day. Intravenous dosing thus may be similar to, or advantageously, may exceed maximal tolerated doses of AmB. Intravenous dosing also may be similar to, or advantageously, may exceed maximal tolerated daily doses of AmB. Intravenous dosing also may be similar to, or advantageously, may exceed maximal tolerated cumulative doses of AmB.

Intravenous dosing also may be similar to, or advantageously, may exceed maximal recommended doses of AmB. Intravenous dosing also may be similar to, or advantageously, may exceed maximal recommended daily doses of AmB. Intravenous dosing also may be similar to, or advantageously, may exceed maximal recommended cumulative doses of AmB.

For any compound described herein the therapeutically effective amount can be initially determined from animal models. A therapeutically effective dose can also be determined from human data for compounds of the invention which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

Amphotericin B is commercially available in a number of formulations, including deoxycholate-based (sometimes referred to as desoxycholate-based) formulations and lipid-based (including liposomal) formulations. Amphotericin B derivative compounds of the invention similarly may be formulated, for example, and without limitation, as deoxycholate-based formulations and lipid-based (including liposomal) formulations.

For use in therapy, an effective amount of the compound of the invention can be administered to a subject by any mode that delivers the compound of the invention to the desired surface. Administering the pharmaceutical composition of the present invention may be accomplished by any means known to the skilled artisan. Routes of administration include but are not limited to oral, intravenous, intramuscular, intraperitoneal, subcutaneous, direct injection (for example, into a tumor or abscess), mucosal, pulmonary (e.g., inhalation), and topical.

For intravenous and other parenteral routes of administration, the compounds of the invention generally may be formulated similarly to AmB. For example, a compound of the invention can be formulated as a lyophilized preparation with deoxycholic acid, as a lyophilized preparation of liposome-intercalated or -encapsulated active compound, as a lipid complex in aqueous suspension, or as a cholesteryl sulfate complex. Lyophilized formulations are generally reconstituted in suitable aqueous solution, e.g., in sterile water or saline, shortly prior to administration.

For oral administration, the compounds (i.e., compounds of the invention, and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4: 185-9 (1982). Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds of the invention (or derivatives thereof). The compound of the invention (or derivative) is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (α1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of compound of the invention (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise compound of the invention (or derivative) dissolved in water at a concentration of about 0.1 to 25 mg of biologically active compound of the invention per mL of solution. The formulation may also include a buffer and a simple sugar (e.g., for compound of the invention stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990), which is incorporated herein by reference.

The compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound of the invention and optionally at least one additional therapeutic agent included in a pharmaceutically acceptable carrier.

The therapeutic agent(s), including specifically but not limited to the compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

### INCORPORATION BY REFERENCE

All U.S. patent application publications and U.S. patents mentioned herein are hereby incorporated by reference in their entirety as if each individual publication or patent was specifically and individually indicated to be incorporated by reference. In case of conflict, the application, including any definitions herein, will control.

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the embodiments described herein is not intended to be limited to the above Description, but rather is as set forth in the appended claims. Those of ordinary skill in the art will appreciate that various changes and modifications to this description may be made without departing from the spirit or scope of the invention, as defined in the following claims.

### Examples

In order that the invention described herein may be more fully understood, the following examples are set forth. The examples described in this application are offered to illustrate the compounds, pharmaceutical compositions, and methods provided herein and are not to be construed in any way as limiting their scope.

### Example 1. Novel Chemical Design with No Mammalian Toxicity

Enabled by the disclosed development of frontier synthesis methods for efficient modification of new AmB derivatives, it is alternatively discovered that AmB primarily kills fungal and human cells by binding ergosterol and cholesterol, respectively; channel formation is not required. All data are consistent with a “sterol sponge” model, whereby AmB self-assembles into a large extramembraneous aggregate and rapidly extracts physiologically vital sterols from fungal and human cells, thereby causing cell death. Frontier SSNMR studies further revealed key insights into the structure of AmB sponge-sterol complexes. Anderson, T. M. et al., Nat Chem Biol 2014, 10 (5), 400-6.

This key discovery opened a path to the rational development of a non-toxic AmB variant. To probe its predicted role in sterol binding, the hydroxyl group was synthetically deleted at the C2′ position on the mycosamine appendage. The resulting derivative, C2′deOAmB, was found to bind ergosterol but, within the detection limits of isothermal titration calorimetry (ITC), not cholesterol. Consistent with the sterol sponge model, this derivative retained good activity against yeast but, most importantly, was nontoxic to human red blood cells and primary (hREC).

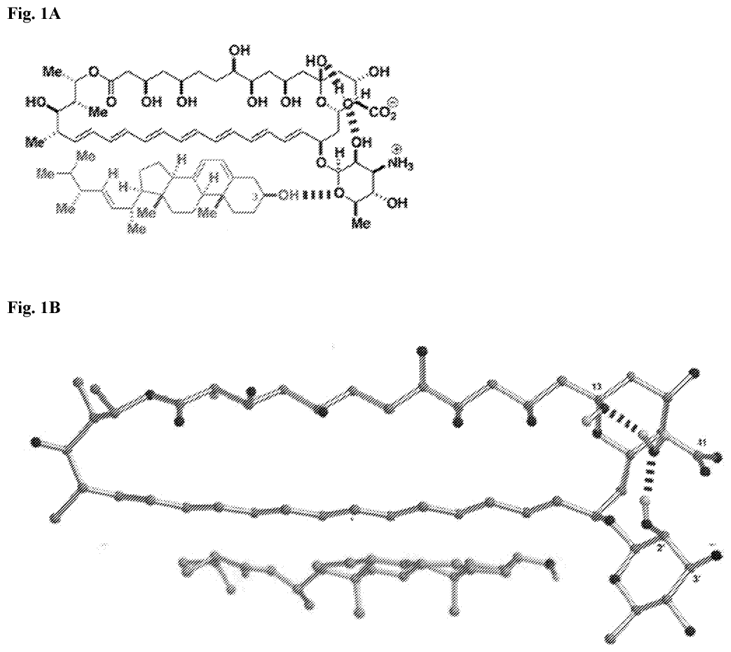

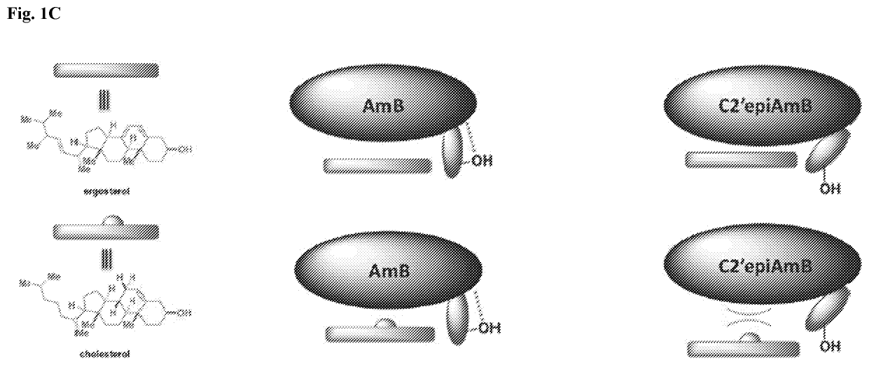

2-Deoxy glycosides are notoriously challenging to synthesize and lack of scalable access to C2′deOAmB has precluded its development. However, these findings led us to a predictive model for guiding the development of more synthetically accessible derivatives with similar selectivity profiles. Crich, D. et. al., The Journal of Organic Chemistry 2011, 76 (22), 9193-9209; Hou, D. et al., Carbohyd Res 2009, 344 (15), 1911-1940; Rodriguez, M. A. Et al., The Journal of Organic Chemistry 2005, 70 (25), 10297-10310; and Hou, D., et al., Organic Letters 2007, 9 (22), 4487-4490. Specifically, to rationalize the selective toxicity of C2′deOAmB for fungal vs. human cells, a model was proposed in which the C2′-OH stabilizes a conformer of AmB that readily binds both ergosterol and cholesterol. The deletion of this hydroxyl group favors a shift to a different conformer or set of conformers which retain the capacity to bind ergosterol but not the more sterically bulky cholesterol. Alternatively, this model suggests that deletion of the C2′OH of AmB causes a small molecule-based allosteric effect that results in ligand-selective binding. Based on the high-resolution X-ray crystal structure of N-iodoacyl AmB (FIG. 1B), there is a prominent water-bridged hydrogen-bond between the hydroxyl groups at C2′ and C13 that may serve to stabilize a particular conformation of the mycosamine appendage relative to the polyene macrolide core. This observation, combined with our previous findings that the mycosamine appendage is critical for binding both ergosterol and cholesterol and observations by SSNMR of direct intermolecular contacts between the AmB polyene and the A/B rings of ergosterol, allowed us to propose a specific structural model for both AmB-sterol complexes consistent with all of our data. Woerly, E. M. et al, Nat Chem 2014, 6 (6), 484-91; Anderson, T. M. et al., Nat Chem Biol 2014, 10 (5), 400-6.

Guided by this model, a simple epimerization of the more synthetically accessible C2′ hydroxyl group, would likewise eliminate the water-bridged C2′OH-C13OH interaction and cause a shift in the orientation of the mycosamine appendage similar to that predicted in C2′deOAmB. The resulting derivative, C2′epiAmB, selectively binds ergosterol and exerts cytocidal action against fungal but not human cells. Notably, C2′epiAmB differs from AmB only in the stereochemistry at a single atom.

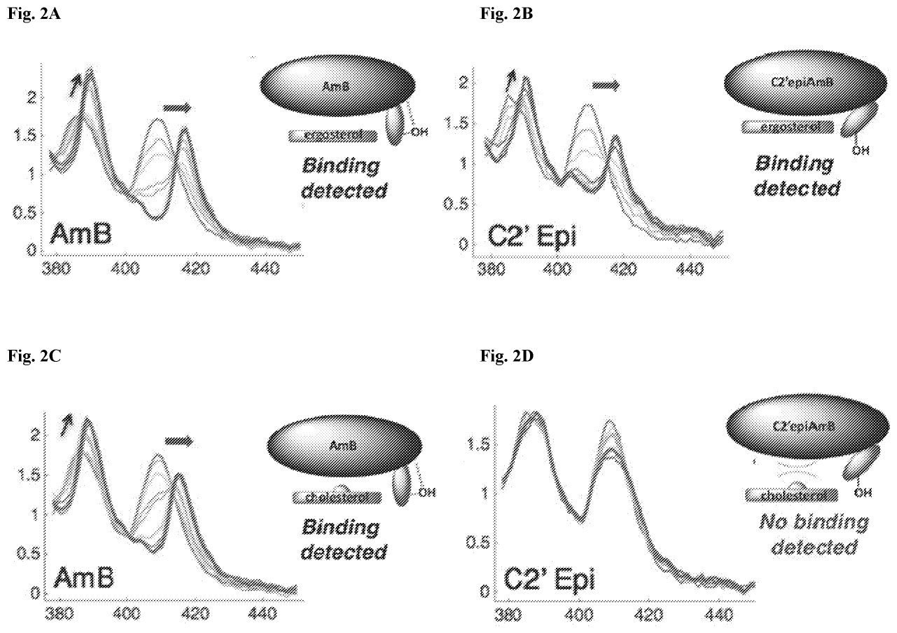

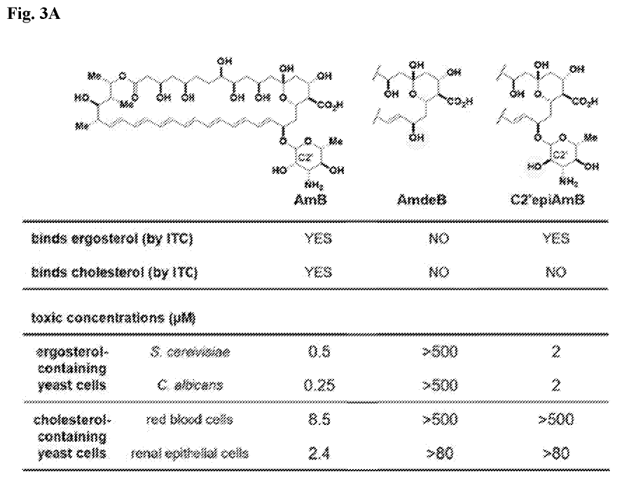

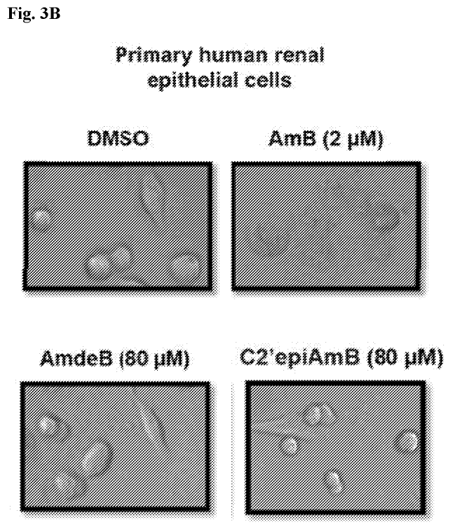

A practical 11-step synthesis of C2′epiAmB using a frontier site-selective acylation method was developed. Wilcock, B. C. et al., Nat Chem 2012, 4 (12), 996-1003; Uno, B. E. A synthesis enabled understanding of Amphotericin B leading to derivatives with improved therapeutic indices. University of Illinois at Urbana-Champaign, 2014. The sterol binding and cell killing activities was then determined. As predicted, like C2′deOAmB, C2′epiAmB was found by ITC to bind ergosterol but not (detectably) cholesterol, and, most importantly, to kill fungal but not human cells (FIGS. 2A-2D, and 3A-3B).

These ITC studies failed to yield S-shaped isotherms, precluding determination of binding constants and other thermodynamic parameters. However, an alternative method was developed for reproducible formation of homogenous AmB and C2′epiAmB sterol sponge aggregates in vitro. Using these preparations, a quantitative UV-Vis and Principle Component (PCA) based assay for determining the apparent Kis for the binding of AmB and C2′epiAmB to ergosterol and cholesterol (FIGS. 2A-2D) was developed. Consistent with the small therapeutic index of this natural product, strong binding of AmB to both ergosterol (K_{D,erg}=120 nM) and cholesterol (K_{D,chol}=840 nM) was observed. Consistent with evaluating C2′epiAmB in vitro, strong binding for C2′epiAmB to ergosterol (K_{D,erg}=150 nM) (FIG. 2B), but little or no binding of cholesterol (FIG. 2D) was observed. The data does not permit confident assigning of a K_{D} for the latter interaction, but it was estimated that it is at least >2000 nM (which is the estimated K_{D,chol} if the data was fitted). Since C2′epiAmB shows no mammalian toxicity, these mechanistically grounded biophysical parameters can be used as benchmarks to prioritize new hybrid derivatives for further development.

### Example 2. AmB Derivatives with No Observed Animal Toxicity

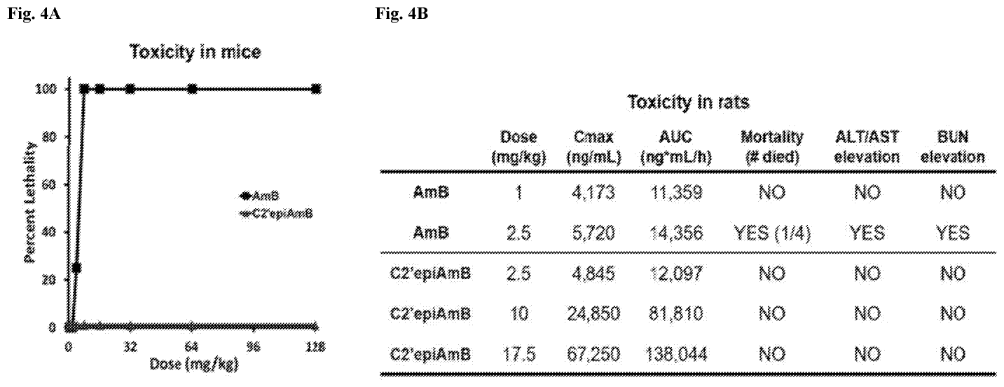

>100 mg of C2′epiAmB was prepared, formulated it as the corresponding deoxycholate complex, and evaluated this derivative head-to-head with AmB-deoxycholate for toxicity and efficacy in animal models. Intravenous (IV) administration of AmB-deoxycholate to mice was found to be lethal at 2-4 mg/kg (FIG. 4A). In contrast, no mortality was observed upon IV injection of C2′epiAmB-deoxycholate even at 128 mg/kg (the highest dose tested). IV administration of AmB-deoxycholate to rats (2.5 mg/kg) caused significant elevations in Blood Urea Nitrogen (BUN), Alanine transaminase/Aspartate transaminase (ALT/AST) and mortality (FIG. 4B). Alternatively, no elevations in BUN or ALT/AST and no mortality when rats were treated with IV injections of C2′ epiAmB at doses of 2, 10, and 17.5 mg/kg (the highest dose that was tested) was observed. The Cₘₐₓ for C2′epiAmB at 17.5 mg/kg was >16-fold higher than the Cₘₐₓ for AmB at 1 mg/kg.

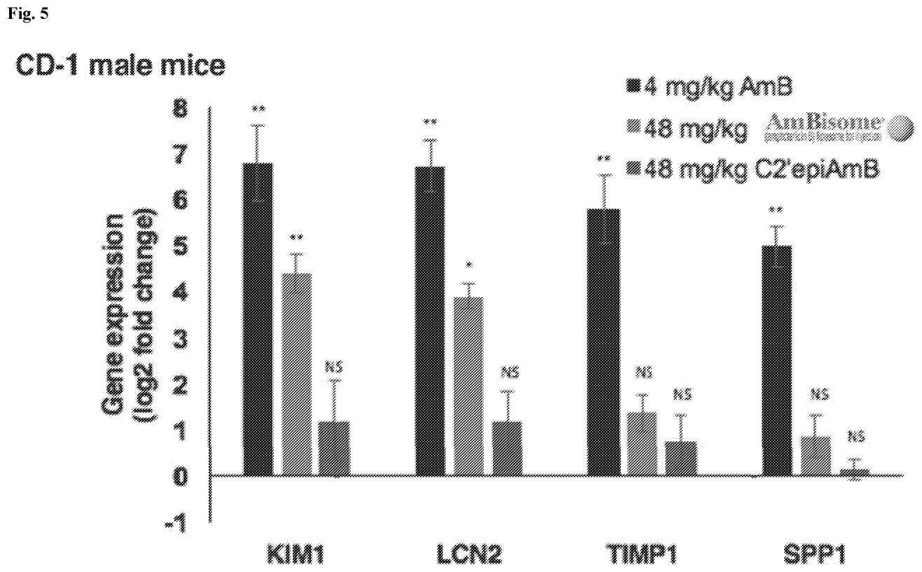

The toxicity of C2′epiAmB to AmBisome®, a liposomal formulation of AmB that is widely used clinically because it is somewhat less toxic than Fungizone® (AmB-deoxycholate) (FIG. 5) was directly compared. Consistent with literature precedent, we confirmed that AmBisome® shows significant toxicity in mice at 48 mg/kg as judged by state-of-the art renal genotoxicity biomarkers. Kondo, C. et al., J Toxicol Sci 2012, 37 (4), 723-37. Alternatively, mice were injected with the same high dose (48 mg/kg) of C2′epiAmB-deoxycholate and observed no significant elevations in these same biomarkers. Thus, C2′epiAmB is significantly less toxic than AmBisome® in mice.

In each case, C2′epiAmB is non-toxic to human red blood cells, primary hREC, mice, and rats up to the highest dose tested. These results are consistent with the finding that, within limits of detection of all of the experiments, C2′epiAmB does not bind cholesterol.

### Example 3. Partially Retained In Vitro Antifungal Activity

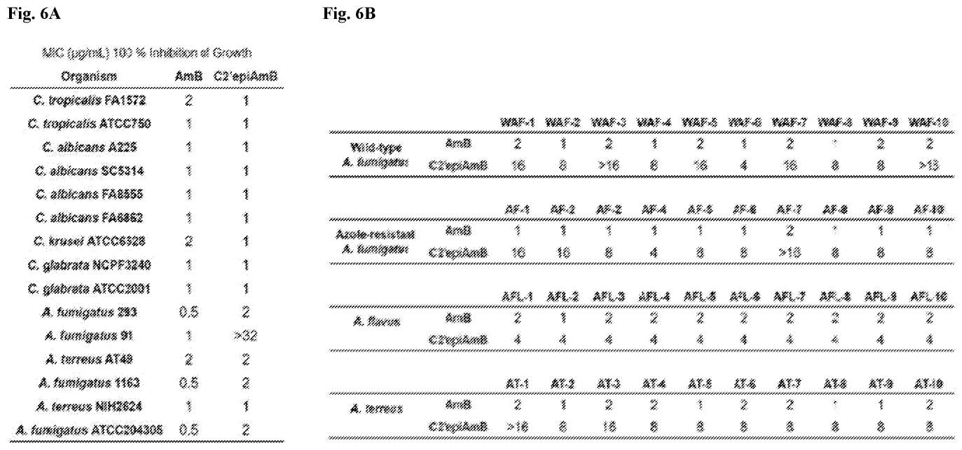

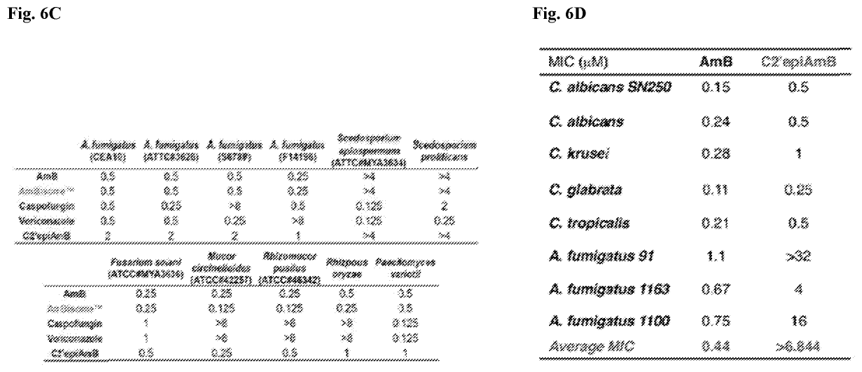

In vitro antifungal activity of C2′ epiAmB was compared with that of AmB against an extensive series of Candida and Aspergillus clinical isolates (FIG. 6A) at Evotec (Oxfordshire, UK). C2′epiAmB showed good activity against many Candida and several Aspergillus strains. However, there were several strains of A. fumigatus (AF293, A1163, and ATC204305), for which C2′epiAmB was 4-fold less potent than AmB, and in one strain (AF91) C2′epiAmB was >32 times less potent. C2′epiAmB was also sent to the US national Fungus Testing Laboratory at UT-San Antonio for antifungal testing against an extended panel of especially challenging 40 Aspergillus clinical isolates, including azole-resistant A. fumigatus, A. flavus, and A. terreus (FIG. 6B). C2′epiAmB was found to be 2-16 times less potent than AmB (average 5.6-fold less potent across all 40 strains). Recently, Steinbach and Burke directly compared the activity of AmB, AmBisome®, caspofungin, voriconazole, and C2′epiAmB against an even broader panel of clinically relevant invasive molds (FIGS. 6C and 6D). These studies again showed good antifungal potency for C2′epiAmB against many strains, including a pan-azole resistant strain (F14196), but also important opportunities for improved activity against Aspergillus.

### Example 4. Retained Primary Mechanism of In Vitro Antifungal Activity

Providing strong evidence for the sterol sponge mechanism, it was previously demonstrated that the antifungal activity of AmB is mitigated via pre-complexing the AmB sterol sponge with ergosterol, thus blocking its ability to extract ergosterol from yeast cells. Anderson, T. M. et al., Nat Chem Biol 2014, 10 (5), 400-6. In a follow-up study performed in collaboration with Susan Lindquist at MIT, this mechanism also showed that it is inherently evasive to clinical resistance, because mutating the ergosterol target causes loss of pathogenicity. Davis, S. A., et al., Nat Chem Biol 2015, 11 (7), 481-7. To test whether C2′epiAmB primarily kills cells via the same sterol sponge mechanism, the C2′epiAmB sponge was similarly pre-complexed with ergosterol. The same reduction in potency for AmB and C2′epiAmB upon ergosterol pre-complexation was observed. Thus, C2′epiAmB similarly kills yeast primarily via sterol binding, and, by extension, the new compounds targeted in this application are expected to have a similar barrier to fungal resistance that has been observed for the past 50+years with AmB.

### Example 5. Non-Toxic Dose-Dependent Efficacy in Murine Invasive Candidiasis

Finally, the dose-dependent efficacy of C2′epiAmB-deoxycholate complex in a murine model of invasive candidiasis was tested. Neutropenic ICR/Swiss mice were injected via lateral tail vein with a lethal inoculum of C. albicans and then treated via single IP injection of AmB-deoxycholate (1 or 4 mg/kg) or C2′epiAmB-deoxycholate (1, 4, 8, or 16 mg/kg). Previous work from the Andes lab shows dose-dependent efficacy for AmB-deoxycholate. Andes, D. et al., Antimicrobial agents and chemotherapy 2001, 45 (3), 922-6. In fact, the PD parameter that best correlates with outcome is Cmax-/MIC. The same was subsequently observed in a pulmonary model of invasive aspergillosis. Wiederhold, N. P. et al., Antimicrobial agents and chemotherapy 2006, 50 (2), 469-73. C2′epiAmB also showed dose-dependent efficacy, with outstanding reductions in fungal burden at the 16 mg/kg dose.

These results show that C2′epiAmB is a unique antifungal agent with potent fungicidal activity against several Candida and Aspergillus strains and no detectable mammalian toxicity, a first for an amphotericin derivative. However, C2′epiAmB also has some important limitations with respect to potency and pathogen scope. Thus, the next plan is to develop a new series of “hybrid” derivatives designed to improve the antifungal potency and pathogen scope of C2′epiAmB while maintaining its lack of toxicity.

### Example 6. Efficacy Studies of C2′EpiAmB in an In Vivo Mouse Model

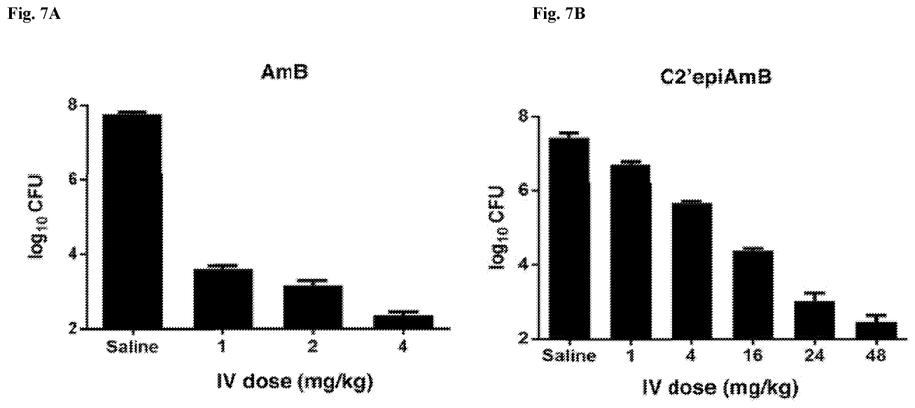

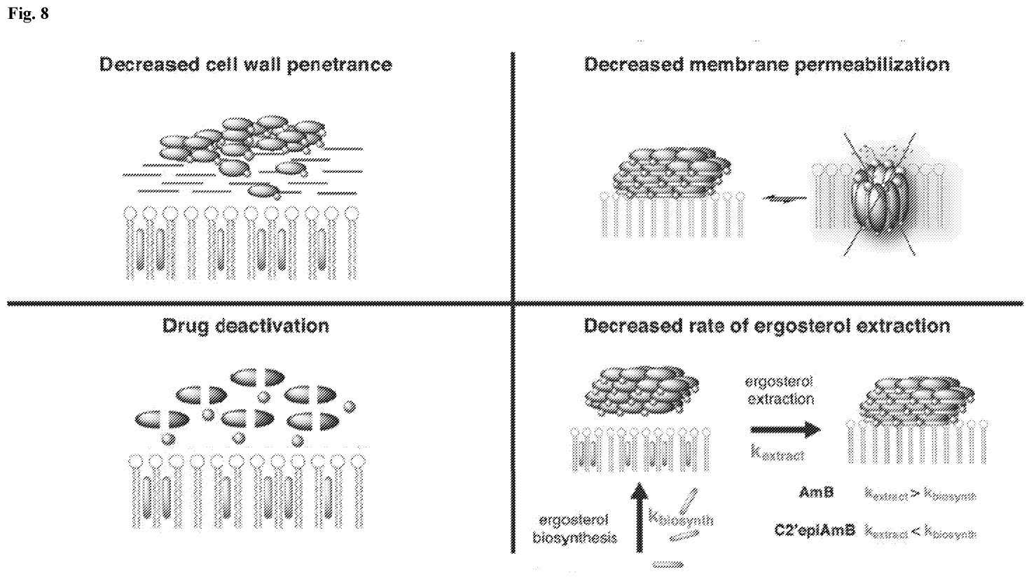

A clinical isolate of C. albicans (SN250) was grown and quantified on SDA. For 24 h before infection, the organism was subcultured at 35° C. on SDA slants. A 106CFU/ml inoculum was prepared by placing six fungal colonies into 5 ml of sterile, depyrogenated normal (0.9%) saline warmed to 35° C. Six week-old ICR/Swiss specific-pathogen-free female mice were obtained from Harlan Sprague Dawley (Madison, Wis.). The mice were weighed (23-27 g) and disseminated candidiasis was induced via tail vein injection of 100 μl of inoculum. AmB and C2′ epiAmB were reconstituted in their deoxycholate form with 1.0 ml of saline5% dextrose. Each animal in the treatment group was given a single 200-μl intravenous (IV) injection of reconstituted AmB and C2′epiAmB 2 h after infection. Doses were calculated in terms of mg of compound per kg of body weight. At 24 h after infection, three animals per experimental condition were killed by CO2 asphyxiation. The kidneys from each animal were removed and homogenized. The homogenate was diluted serially tenfold with 9% saline and plated on SDA. The plates were incubated for 24 h at 35° C. and inspected for CFU viable counts. The lower limit of detection for this technique is 100 CFU/ml. All of the results are expressed as the mean log 10 CFU per kidney for three animals, and are shown in FIG. 7A and FIG. 7B.

### Example 7. Study of Rate of Ergosterol Extraction

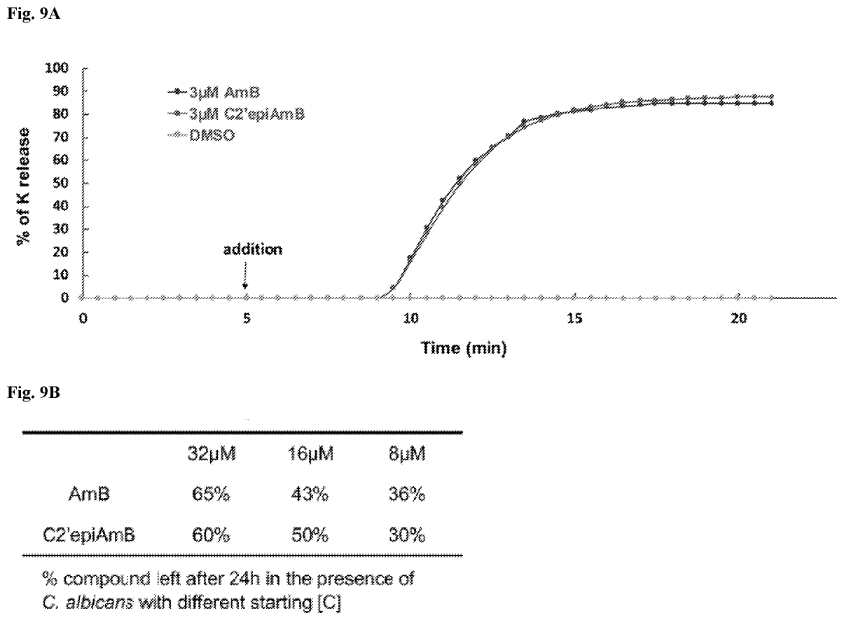

Potassium efflux assay protocol: An overnight culture of Candida SN250 in YPD was centrifuged at 300 g for 5 minutes at 23° C. The supernatant was decanted and the cells were washed twice with sterile water. After the second wash step, the cells were suspended in 150 mM NaCl, 5 mM HEPES pH 7.4 (Na buffer) to an OD600 of 1.5 (1×10⁹ CFU/mL) as measured by a Shimadzu (Kyoto, Japan) PharmaSpec UV-1700 UV/Vis spectrophotometer. A 3 mL sample of the cell suspension was taken to a glass vial held in aluminum block with stirring for approximately 10 minutes before data collection. The probe was then inserted and data was collected for 5 minutes before adding 30 μL of the compound in question as a 0.3 mM solution in DMSO. The cell suspension was stirred and data were collected for 30 minutes and then 30 μL of a 1% aqueous solution of digitonin was added to effect complete potassium release and data were collected for an additional 15 minutes. The results of these experiments are shown in FIG. 9A and FIG. 9B.

Stability check by HPLC protocol: After the reading of MIC of AmB and C2′epiAmB, 50 μL of aliquot from different starting concentration of AmB/C2′epiAmB was injected to HPLC and the area under the curve was used to determine the remaining concentration of compound via pre-established standard curve. Then remaining concentration was divided by the starting concentration to get the percentage remaining of compound.

Ergosterol extraction and yeast remaining assay protocol: 750-ml overnight cultures of Candida SN250 were grown to stationary phase (0D600 of ˜1.7 as measured with a Shimadzu PharmaSpec UV-1700 UV-vis spectrophotometer). This culture was divided equally into 50-ml Falcon centrifuge tubes. Stock solutions of AmB and C2′epiAmB were prepared in DMSO. Cells were treated with either a DMSO-only control, 5 μM AmB and C2′epiAmB for 10 min and 30 min. Treated tubes were incubated on the rotary shaker (200 r.p.m.) at 35° C. for the time of exposure. To quantify colony-forming units (CFUs), at the end of exposure, aliquots were taken from the samples, diluted and plated on SDA plates. The plates were then incubated for 24 h at 35° C., and colony-forming units were counted. To quantify the percentage of Erg remaining, yeast membranes were isolated using a modified version of Haas' spheroplasting and isosmotic cell lysis protocol and simple differential ultracentrifugation. At the end of the exposure time, tubes were removed from the shaker and centrifuged for 5 min at 3,000 g at room temperature. The supernatant was decanted, and 5 ml of wash buffer (dH2O, 1 M DTT, 1 M Tris-HCl, pH 9.4) was added. The tubes were vortexed to resuspend and incubated in a 30° C. water bath for 10 min. Tubes were then centrifuged again for 5 min at 3,000 g, and the supernatant was decanted. 1 ml of spheroplasting buffer (1M KPi, YPD medium, 4 M Sorbitol) and 100 μl of a 5 mg/ml solution of lyticase from Arthrobacter luteus (L2524 Sigma-Aldrich) was added to each tube, and each tube was then vortexed to resuspend. Tubes were incubated in a 30° C. water bath for 30 min, with occasional swirling. After incubation, tubes were centrifuged for 10 min at 1,080 g at 4° C., and the supernatant was decanted. 1 ml of PBS buffer and 20 μl of a 0.4 mg/ml dextran in 8% Ficoll solution was added to each tube and mixed very gently to resuspend. This suspension was placed on ice for 4 min and then heat shocked in a 30° C. water bath for 3 min. The suspensions were then transferred to Eppendorf tubes, vortexed to ensure complete lysis, and centrifuged at 15,000 g at 4° C. for 15 min to remove unlysed cells and cell debris. The resulting supernatants were transferred to thick-wall polycarbonate ultracentrifuge tubes (3.5 ml, 13×51 mm, 349622 Beckman Coulter) and spun for 1 h at 100,000 g at 4° C. in a Beckman Coulter TLA-100.3 fixed-angle rotor in a Beckman TL-100 ultracentrifuge. The supernatant was poured off. The remaining membrane pellet was resuspended in 1 ml PBS buffer and stored at −80° C. until further analysis. The results of these experiments are shown in FIG. 11A and FIG. 11B.

Gas chromatography quantification of sterols. 750 μl of each membrane pellet sample and 20 μl of internal standard (4 mg/ml cholesterol in chloroform) were dissolved in 3 ml 2.5% ethanolic KOH in a 7-ml vial, which was then vortexed gently, capped and heated in a heat block on a hot plate at 90° C. for 1 h. The vials were then removed from the heat source and allowed to cool to room temperature. 1 ml of brine was added to the contents of each vial. Extraction was performed twice, each with 3 ml of hexane. Organic layers were removed in both extractions, dried over magnesium sulfate, filtered through Celite 545 (Sigma-Aldrich) and transferred to another 7 ml vial. The contents of the vial were then concentrated in vacuo in a 30° C. water bath. The resulting sterol films were resuspended in 100 μl pyridine and 100 μl N,O-bis-(trimethylsilyl)-trifluoroacetamide with 1% trimethylchlorosilane (T6381-10AMP Sigma-Aldrich) by vortexing gently. This solution was heated at 60° C. for 1 h. The vials were placed on ice, and the solvent was evaporated off by nitrogen stream. Vials must be kept at a low temperature to prevent evaporation of the sterol TMS ethers along with the solvent. The resulting films were resuspended in 100 μl of decane, filtered and transferred to a GC vial insert for analysis. Gas chromatography analysis was carried out on an Agilent 7890A gas chromatograph equipped with a FID, an Agilent GC 7693 Autosampler and a Dell computer running Microsoft XP that uses ChemStation v.B.04.02 SP1. Samples were separated on a 30-m, 0.320-mm ID, 0.25 μm film HP-5 capillary column (19091J-413 Agilent) using hydrogen as a carrier gas with an average velocity of 84.8 cm/s. Nitrogen make-up gas, hydrogen and compressed air were used for the FID. A split/splitless injector was used in a 20:1 split. The injector volume was 2 μl. The column temperature was initially held at 250° C. for 0.5 min and then was ramped to 265° C. at a rate of 10° C./min, with a final hold time of 12.5 min. The injector and detector temperature were maintained at 270° C. and 290° C., respectively. The value reported for each time point was calculated by dividing the value for the treatment group by the value for the DMSO control at the same time point and then normalizing the DMSO control to 100%.

### Example 8. Killing Kinetics for C2′epiAmB as Compared to AmB or DMSO

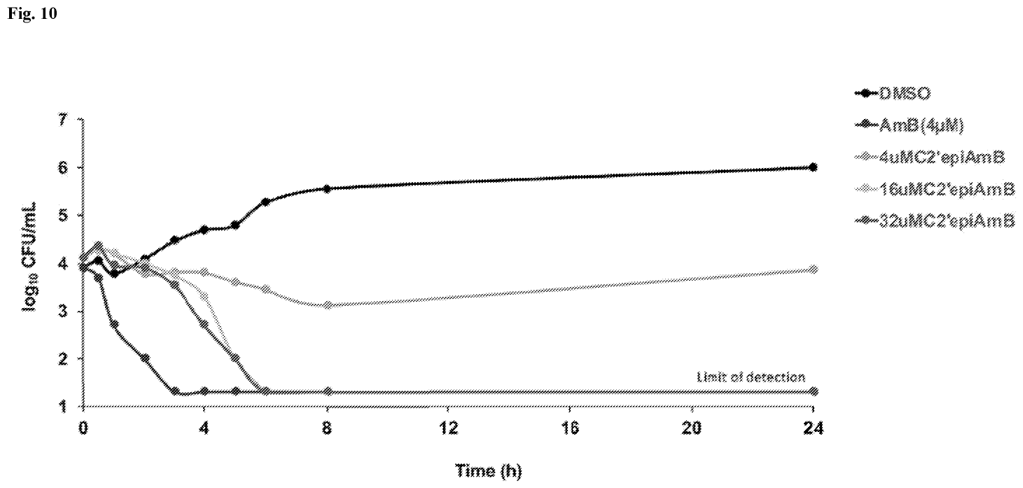

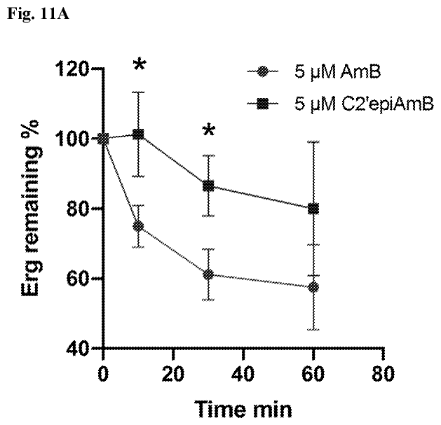

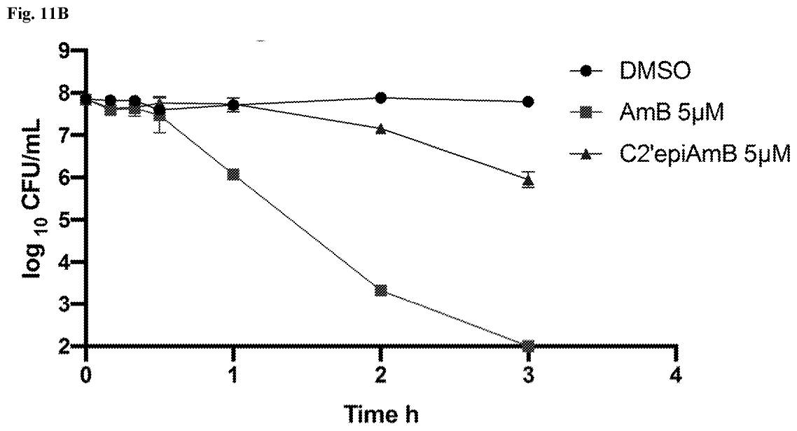

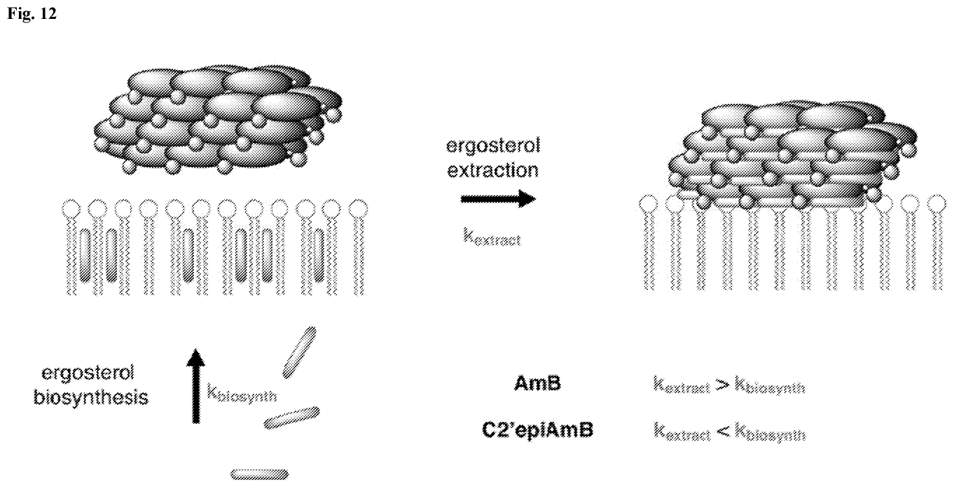

50 mL of YPD media was inoculated and incubated overnight in a shaker incubator. The cell suspension was then diluted with YPD to an OD600 of 0.10 (˜5×10⁵ cfu/mL) as measured by a Shimadzu (Kyoto, Japan) PharmaSpec UV-1700 UV/Vis spectrophotometer. The solution was diluted 10-fold with YPD, and 990 μL aliquots of the dilute cell suspension were added to sterile 1.7 mL eppendorf tubes. Compounds were prepared as stock solution in DMSO. The addition of 10 μL of test compound to the dilute cell suspension made a final concentration 100-fold dilution of each compound stock solution. The concentration of DMSO in each eppendorf tube was 1% and a control sample to confirm viability using only 1% DMSO was also performed. The samples were vortexed and incubated at 35° C. for 24 hours. At predetermined time points (0, 0.5, 1, 2, 3, 4, 5, 6, 8 and 24 h), a 10 μL sample was removed from each tube and serially diluted 10 fold with YPD, and a 10 μL aliquot was plated onto a YPD plate for colony count determination. When colony counts were expected to be less than 1,000 CFU/mL, a 50 μL aliquot was taken directly from the test solution and plated onto a YPD plate without dilution. Plates were incubated at 35° C. for 24 hours prior to examination. The results of this experiment are shown in FIG. 10.

### Example 9. Killing Kinetics for Combination of C2′epiAmB and Ketoconazole

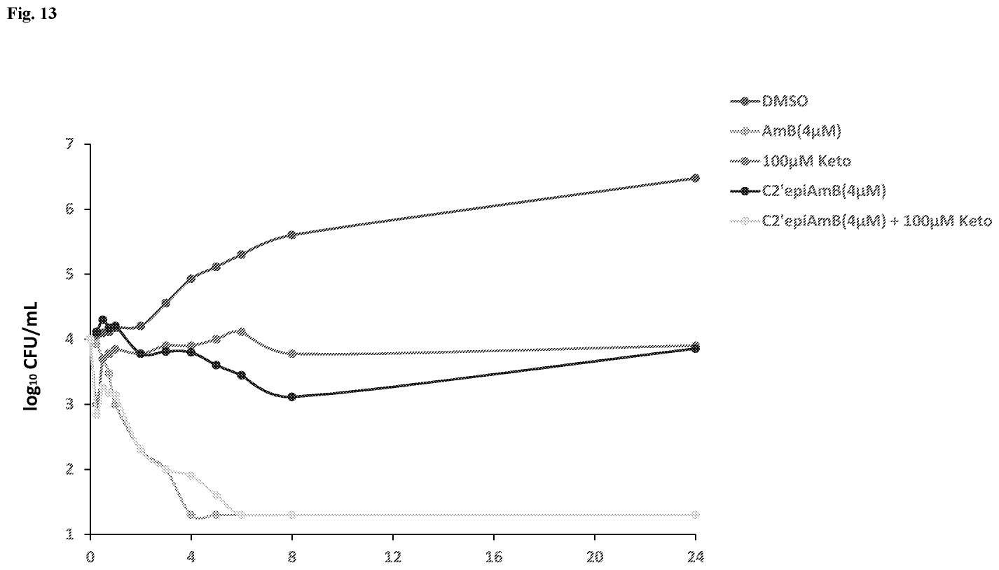

50 mL of YPD media was inoculated and incubated overnight in a shaker incubator. The cell suspension was then diluted with YPD to an OD600 of 0.10 (˜5×10⁵ cfu/mL) as measured by a Shimadzu (Kyoto, Japan) PharmaSpec UV-1700 UV/Vis spectrophotometer. The solution was diluted 10-fold with YPD, and 990 μL aliquots of the dilute cell suspension were added to sterile 1.7 mL eppendorf tubes. Compounds were prepared as stock solution in DMSO. The addition of 10 μL of test compound to the dilute cell suspension made a final concentration 100-fold dilution of each compound stock solution. For cotreatment, 5 μL of C2′epiAmB and 54, ketoconazole solution with 200× of the final concentration were added to the eppendorf tubes. The concentration of DMSO in each eppendorf tube was 1% and a control sample to confirm viability using only 1% DMSO was also performed. The samples were vortexed and incubated at 35° C. for 24 hours. At predetermined time points (0, 0.5, 1, 2, 3, 4, 5, 6, 8, and 24 h), a 10₁..t.L sample was removed from each tube and serially diluted 10 fold with YPD, and a 10μ.L aliquot was plated onto a YPD plate for colony count determination. When colony counts were expected to be less than 1,000 CFU/mL, a 50μ.L aliquot was taken directly from the test solution and plated onto a YPD plate without dilution. Plates were incubated at 35° C. for 24 hours prior to examination. The results of this experiment are shown in FIG. 13.

### Example 10. Clinical Isolates Including Candida and Aspergillus with High MICs with

azoles (>4 μg/mL).

| Isolate | | | | | | |
|---|---|---|---|---|---|---|
| source | | | | | | |
| Collection | | | | City or | Medical | Infection |
| No. | Organism | Year | Country | State | Service | Source |
| ATCC | A. flavus | 2018 | — | — | — | — |
| 204304 | | | | | | |
| 1085986 | A. flavus SC | 2018 | USA | Michigan | Internal | Bronchoalveolar |
| | | | | | Medicine | lavage |
| 1077391 | A. fumigatus | 2018 | Italy | Milan | Ambulatory/ | Invasive |
| | | | | | Outpatient | Pulmonary |
| ATCC | A. fumigatus | 2018 | — | — | — | — |
| MYA- | | | | | | |
| 3626 | | | | | | |
| 1087597 | F. | 2018 | USA | Virginia | Internal | Blood |
| | oxysporum | | | | Medicine | culture |
| | SC | | | | | |
| 1047542 | F. solani SC | 2018 | USA | Utah | Intensive Care | Blood |
| | | | | | Unit | culture |
| 967374 | C. albicans | 2016 | USA | California | | Other |
| 1081160 | C. auris | 2018 | USA | New York | Surgery | Blood |
| | | | | | | culture |
| 1046695 | C. glabrata | 2018 | USA | Kansas | Cardiothoracic/ | Blood |
| | | | | | Pulmonary | culture |
| 975691 | C. krusei | 2016 | USA | Washington | Hematology/ | Blood |
| | | | | | Oncology | culture |
| ATCC | C. krusei | 2018 | — | — | — | — |
| 6258 | | | | | | |
| 1048737 | C. | 2018 | USA | Ohio | Transplant | Blood |
| | parapsilosis | | | | | culture |
| ATCC | C. | 2018 | — | — | — | — |
| 22019 | parapsilosis | | | | | |
| 1046709 | C. tropicalis | 2018 | USA | Kansas | Neurology | Wound/ |
| | | | | | | Drainage/ |
| | | | | | | Ulcer |

### Example 11. Checkerboard Assay for Synergy Between AmB/C2′epiAmB and 3 Azoles Including Ketoconazole, Fluconazole, and Voriconazole

Isolates were tested against varying concentrations of amphotericin B and C2′epiAmB (range, 0.06 to 4 mg/L) and combined with fluconazole (range, 0.12 to 128 mg/L) or ketoconazole (range 0.008 to 8 mg/L) or voriconazole (range, 0.004 to 4 mg/L) using a checkerboard grid design following the Clinical and Laboratory Standards Institute (CLSI) broth microdilution method (M23Ed5).

TABLE 1
Checkerboard assay results for AmB and ketoconazole.

| | | | | MIC of AmB (mg/L) when |
|---|---|---|---|---|
| Isolate Source | | MIC (mg/L) | | added 1 log2 unit lower of |
| Collection no. | Organism | Ketoconazole | AmB | azoles' MIC |
| ATCC | A. flavus | 2 | 1 | 1 |
| 204304 | | | | |
| 1085986 | A. flavus SC | 4 | 2 | 1 |
| 1077391 | A. fumigatus | >8 | 1 | 0.06 |
| ATCC MYA- | A. fumigatus | 8 | 1 | 2 |
| 3626 | | | | |
| 1087597 | F. oxysporum | >8 | 2 | 0.06 |
| | SC | | | |
| 1047542 | F. solani SC | >8 | 2 | 0.06 |
| 967374 | C. albicans | 0.25 | 0.5 | 0.5 |
| 1081160 | C. auris | 0.5 | 1 | 1 |
| 1046695 | C. glabrata | 4 | 0.5 | 0.06 |
| 975691 | C. krusei | 1 | 1 | 0.5 |
| ATCC 6258 | C. krusei | 0.25 | 0.5 | 0.5 |
| 1048737 | C. parapsilosis | 0.015 | 0.5 | 0.25 |
| ATCC 22019 | C. parapsilosis | 0.06 | 0.5 | 0.25 |
| 1046709 | C. tropicalis | 0.12 | 0.5 | 0.5 |

TABLE 2
Checkerboard assay results for AmB and fluconazole.

| | | | | MIC of AmB (mg/L) when |
|---|---|---|---|---|
| Isolate Source | | MIC (mg/L) | | added 1 log2 unit lower of |
| Collection no. | Organism | Fluconazole | AmB | azoles' MIC |
| ATCC 204304 | A. flavus | — | — | |
| 1085986 | A. flavus SC | — | — | |
| 1077391 | A. fumigatus | — | — | |
| ATCC MYA- | A. fumigatus | — | — | |
| 3626 | | | | |
| 1087597 | F. oxysporum | — | — | |
| | SC | | | |
| 1047542 | F. solani SC | — | — | |
| 967374 | C. albicans | 16 | 1 | 0.5 |
| 1081160 | C. auris | >128 | 1 | 0.06 |
| 1046695 | C. glabrata | 128 | 0.5 | 0.5 |
| 975691 | C. krusei | 128 | 1 | 0.5 |
| ATCC 6258 | C. krusei | 32 | 0.5 | 0.5 |
| 1048737 | C. parapsilosis | 0.5 | 0.5 | 0.25 |
| ATCC 22019 | C. parapsilosis | 1 | 0.25 | 0.25 |
| 1046709 | C. tropicalis | 4 | 0.5 | 0.25 |

TABLE 3
Checkerboard assay results for AmB and voriconazole.

| | | | | MIC of AmB (mg/L) when added |
|---|---|---|---|---|
| Isolate Source | | MIC (mg/L) | | 1 log2 unit lower of |
| Collection no. | Organism | Voriconazole | AmB | azoles' MIC |
| ATCC 204304 | A. flavus | 1 | 1 | 0.5 |
| 1085986 | A. flavus SC | 1 | 2 | 2 |
| 1077391 | A. fumigatus | 2 | 1 | 1 |
| ATCC MYA- | A. fumigatus | 0.5 | 1 | 1 |
| 3626 | | | | |
| 1087597 | F. oxysporum SC | 4 | 2 | 2 |
| 1047542 | F. solani SC | 4 | 2 | 2 |
| 967374 | C. albicans | 0.25 | 1 | 0.5 |
| 1081160 | C. auris | 1 | 1 | 1 |
| 1046695 | C. glabrata | 4 | 0.5 | 0.5 |
| 975691 | C. krusei | 4 | 0.5 | 0.5 |
| ATCC 6258 | C. krusei | 0.25 | 1 | 0.5 |
| 1048737 | C. parapsilosis | 0.008 | 0.5 | 0.25 |
| ATCC 22019 | C. parapsilosis | 0.03 | 1 | 0.5 |
| 1046709 | C. tropicalis | 0.12 | 0.5 | 0.5 |

TABLE 4
Checkerboard assay results for C2′epiAmB and ketoconazole.

| | | MIC (mg/L) | | MIC of C2′epiAmB (mg/L) |
|---|---|---|---|---|
| Isolate Source | | | C2′epi | when added 1 log2 unit lower |
| Collection no. | Organism | Ketoconazole | AmB | of azoles' MIC |
| ATCC | A. flavus | 2 | 2 | 2 |
| 204304 | | | | |
| 1085986 | A. flavus SC | 4 | 4 | >4 |
| 1077391 | A. fumigatus | >8 | 4 | 0.06 |
| ATCC MYA- | A. fumigatus | >8 | >4 | 0.06 |
| 3626 | | | | |
| 1087597 | F. oxysporum | >8 | >4 | 0.06 |
| | SC | | | |
| 1047542 | F. solani SC | >8 | >4 | 0.06 |
| 967374 | C. albicans | 0.25 | 2 | 1 |
| 1081160 | C. auris | 0.5 | 2 | 2 |
| 1046695 | C. glabrata | 4 | 1 | 1 |
| 975691 | C. krusei | 2 | 2 | 2 |
| ATCC 6258 | C. krusei | 0.25 | 2 | 1 |
| 1048737 | C. | 0.03 | 1 | 0.5 |
| | parapsilosis | | | |
| ATCC 22019 | C. | 0.06 | 1 | 0.5 |
| | parapsilosis | | | |
| 1046709 | C. tropicalis | 0.12 | 1 | 1 |

TABLE 5
Checkerboard assay results for C2′epiAmB and fluconazole.

| | | MIC(mg/L) | | MIC of C2′epiAmB (mg/L) |
|---|---|---|---|---|
| Isolate Source | | | C2′epi | when added 1 log2 unit lower |
| Collection no. | Organism | Fluconazole | AmB | of azoles' MIC |
| ATCC 204304 | A. flavus | — | — | |
| 1085986 | A. flavus SC | — | — | |
| 1077391 | A. fumigatus | — | — | |
| ATCC MYA- | A. fumigatus | — | — | |
| 3626 | | | | |
| 1087597 | F. oxysporum SC | — | — | |
| 1047542 | F. solani SC | — | — | |
| 967374 | C. albicans | 16 | 2 | 2 |
| 1081160 | C. auris | >128 | 4 | 0.06 |
| 1046695 | C. glabrata | 128 | 2 | 2 |
| 975691 | C. krusei | 128 | 2 | 2 |
| ATCC 6258 | C. krusei | 32 | 2 | 2 |
| 1048737 | C. parapsilosis | 1 | 2 | 1 |
| ATCC 22019 | C. parapsilosis | 2 | 2 | 1 |
| 1046709 | C. tropicalis | 4 | 1 | 1 |

TABLE 6
Checkerboard assay results for C2′epiAmB and voriconazole.

| | | MIC (mg/L) | | MIC of C2′epiAmB (mg/L) |
|---|---|---|---|---|
| Isolate Source | | | C2′epi | when added 1 log2 unit lower |
| Collection no. | Organism | Voriconazole | AmB | of azoles' MIC |
| ATCC 204304 | A. flavus | 1 | 2 | 1 |
| 1085986 | A. flavus SC | 1 | 4 | >4 |
| 1077391 | A. fumigatus | 2 | 4 | >4 |
| ATCC MYA- | A. fumigatus | 0.5 | 4 | >4 |
| 3626 | | | | |
| 1087597 | F. oxysporum SC | 4 | >4 | >4 |
| 1047542 | F. solani SC | 4 | >4 | >4 |
| 967374 | C. albicans | 0.25 | 2 | 1 |
| 1081160 | C. auris | 1 | 4 | 2 |
| 1046695 | C. glabrata | 4 | 1 | 1 |
| 975691 | C. krusei | 4 | 2 | 2 |
| ATCC 6258 | C. krusei | 0.25 | 1 | 1 |
| 1048737 | C. parapsilosis | 0.015 | 1 | 0.5 |
| ATCC 22019 | C. parapsilosis | 0.03 | 1 | 1 |
| 1046709 | C. tropicalis | 0.12 | 1 | 1 |

We claim:

1. A pharmaceutical composition, comprising a polyene macrolide antibiotic; and an azole antifungal compound; wherein the azole antifungal compound inhibits lanosterol 14-alpha demethylase.

2. The pharmaceutical composition of claim 1, wherein the azole antifungal compound is an imidazole, a triazole, or a thiazole.

3. The pharmaceutical composition of claim 2, wherein the azole antifungal compound is an imidazole.

4. The pharmaceutical composition of claim 3, wherein the imidazole is selected from the group consisting of bifonazole, butoconazole, clotrimazole, econazole, fenticonazole, isoconazole, ketoconazole, luliconazole, miconazole, omoconazole, oxiconazole, sertaconazole, sulconazole, and tioconazole.

5. The pharmaceutical composition of claim 2, wherein the azole antifungal compound is a triazole.

6. The pharmaceutical composition of claim 5, wherein the triazole is selected from the group consisting of albaconazole, efinaconazole, epoxiconazole, fluconazole, isavuconazole, itraconazole, posaconazole, propiconazole, ravuconazole, terconazole, and voriconazole.

7. The pharmaceutical composition of claim 2, wherein the azole antifungal compound is a thiazole.

8. The pharmaceutical composition of claim 7, wherein the thiazole is abafungin.

9. The pharmaceutical composition of any one of claims 1-8, wherein the polyene macrolide antibiotic is selected from the group consisting of amphotericin B, C2′epi amphotericin B, candicidin, filipin, hamycin, natamycin, nystatin, and rimocidin.

10. The pharmaceutical composition of claim 9, wherein the polyene macrolide antibiotic is amphotericin B.

11. The pharmaceutical composition of claim 9, wherein the polyene macrolide antibiotic is C2′epi amphotericin B.

12. The pharmaceutical composition of any one of claims 1-11, wherein the composition is an intravenous dosage form.

13. The pharmaceutical composition of any one of claims 1-11, wherein the composition is an oral dosage form.

14. The pharmaceutical composition of claim 13, wherein the oral dosage form is a tablet.

15. The pharmaceutical composition of claim 13, wherein the oral dosage form is a capsule.

16. A method of treating a systemic fungal infection, comprising co-administering to a mammal in need thereof an effective amount of a polyene macrolide antibiotic; and an effective amount of an azole antifungal compound; wherein the azole antifungal compound inhibits lanosterol 14-alpha demethylase.

17. The method of claim 16, wherein the azole antifungal compound is an imidazole, a triazole, or a thiazole.

18. The method of claim 17, wherein the azole antifungal compound is an imidazole.

19. The method of claim 18, wherein the imidazole is selected from the group consisting of bifonazole, butoconazole, clotrimazole, econazole, fenticonazole, isoconazole, ketoconazole, luliconazole, miconazole, omoconazole, oxiconazole, sertaconazole, sulconazole, and tioconazole.

20. The method of claim 17, wherein the azole antifungal compound is a triazole.

21. The method of claim 20, wherein the triazole is selected from the group consisting of albaconazole, efinaconazole, epoxiconazole, fluconazole, isavuconazole, itraconazole, posaconazole, propiconazole, ravuconazole, terconazole, and voriconazole.

22. The method of claim 17, wherein the azole antifungal compound is a thiazole.

23. The method of claim 22, wherein the thiazole is abafungin.

24. The method of any one of claims 16-23, wherein the polyene macrolide antibiotic is selected from the group consisting of amphotericin B, C2′epi amphotericin B, candicidin, filipin, hamycin, natamycin, nystatin, and rimocidin.

25. The method of claim 24, wherein the polyene macrolide antibiotic is amphotericin B.

26. The method of claim 24, wherein the polyene macrolide antibiotic is C2′epi amphotericin B.

27. The method of any one of claims 16-26, wherein the polyene macrolide antibiotic and the azole antifungal compound are administered in separate dosage forms.

28. The method of claim 27, wherein the polyene macrolide antibiotic and the azole antifungal compound are administered simultaneously, sequentially, or intermittently.

29. The method of claim 28, wherein the polyene macrolide antibiotic and the azole antifungal compound are administered simultaneously.

30. The method of claim 28, wherein the polyene macrolide antibiotic and the azole antifungal compound are administered sequentially.

31. The method of claim 28, wherein the polyene macrolide antibiotic and the azole antifungal compound are administered intermittently.

32. The method of any one of claims 16-26, wherein the polyene macrolide antibiotic and the azole antifungal compound are administered in a combined dosage form.

33. The method of claim 32, wherein the combined dosage form is administered intravenously.

34. The method of claim 32, wherein the combined dosage form is administered orally.

35. A packaged pharmaceutical product, comprising a polyene macrolide antibiotic; and an azole antifungal compound; wherein the azole antifungal compound inhibits lanosterol 14-alpha demethylase.

36. The packaged pharmaceutical product of claim 35, wherein the azole antifungal compound is an imidazole, a triazole, or a thiazole.

37. The packaged pharmaceutical product of claim 36, wherein the azole antifungal compound is an imidazole.

38. The packaged pharmaceutical product of claim 37, wherein the imidazole is selected from the group consisting of bifonazole, butoconazole, clotrimazole, econazole, fenticonazole, isoconazole, ketoconazole, luliconazole, miconazole, omoconazole, oxiconazole, sertaconazole, sulconazole, and tioconazole.

39. The packaged pharmaceutical product of claim 36, wherein the azole antifungal compound is a triazole.

40. The packaged pharmaceutical product of claim 39, wherein the triazole is selected from the group consisting of albaconazole, efinaconazole, epoxiconazole, fluconazole, isavuconazole, itraconazole, posaconazole, propiconazole, ravuconazole, terconazole, and voriconazole.

41. The packaged pharmaceutical product of claim 36, wherein the azole antifungal compound is a thiazole.

42. The packaged pharmaceutical product of claim 41, wherein the thiazole is abafungin.

43. The packaged pharmaceutical product of any one of claims 35-42, wherein the polyene macrolide antibiotic is selected from the group consisting of amphotericin B, C2′epi amphotericin B, candicidin, filipin, hamycin, natamycin, nystatin, and rimocidin.

44. The packaged pharmaceutical product of claim 43, wherein the polyene macrolide antibiotic is amphotericin B.

45. The packaged pharmaceutical product of claim 43, wherein the polyene macrolide antibiotic is C2′epi amphotericin B.

46. The packaged pharmaceutical product of any one of claims 35-45, wherein the polyene macrolide antibiotic and the azole antifungal compound are in separate dosage forms.

47. The packaged pharmaceutical product of any one of claims 35-45, wherein the polyene macrolide antibiotic and the azole antifungal compound are in a combined dosage form.

48. The packaged pharmaceutical product of claim 47, wherein the combined dosage form is an intravenous dosage form.

49. The packaged pharmaceutical product of claim 47, wherein the combined dosage form is an oral dosage form.
